(19) United States
(12) Patent Application Publication
(10) Pub. No.: US 2016/0354304 A1
(43) Pub. Date: Dec. 8, 2016

(54) Lip Treatment Composition

(71) Applicant: Ronald Anderson, Sylmar, CA (US)

(72) Inventor: Ronald Anderson, Sylmar, CA (US)

(21) Appl. No.: 15/238,906

(22) Filed: Aug. 17, 2016

Related U.S. Application Data
(63) Continuation-in-part of application No. 14/738,002, filed on Jun. 12, 2015, now abandoned.
(60) Provisional application No. 62/012,730, filed on Jun. 16, 2014.

(51) Int. Cl. A61K 8/97; A61Q 19/00; A61K 8/92

(52) U.S. Cl. CPC A61K 8/97; A61K 8/925; A61Q 19/001

(57) ABSTRACT

A lip treatment composition for creating a perceptible buzzing sensation when applied to mucosa membrane of a user. The composition has one or more base ingredients and at least one active ingredient comprising Zanthoxylum essential oil extracted in a liquid carbon dioxide process that induces a perceptible sensation reminiscent of electrical buzzing when applied to the mucosa membrane of the user. The Zanthoxylum essential oil could comprises between approximately 2.0000% and 4.0000% by weight of the composition and could comprise approximately 3.3000% by weight of the composition. The lip treatment composition can be formed under a process including heating the one or more base ingredients, adding the at least one active ingredient, and mixing the one or more base ingredients and the at least one active ingredient.

### RELATED APPLICATION

This application is a continuation-in-part of U.S. patent application Ser. No. 14/738,002, filed Jun. 12, 2015, which claimed priority to Provisional Application No. 62/012,730, filed Jun. 16, 2014, both of which being incorporated herein by reference.

### FIELD OF THE INVENTION

The present invention relates generally to lip treatment products. Stated more particularly, disclosed herein is a lip treatment composition that, when applied directly to the lips, causes a perceptible sensation, such as a tingling sensation reminiscent of electrical buzzing and pulsing, across the surface of the lips.

### BACKGROUND OF THE INVENTION

Numerous pomades for lips have been disclosed by the prior art. Indeed, new disclosures for lip balms, lip gloss, and lipsticks are continually added to the multi-billion dollar cosmetics industry. By way of example and not limitation, lip balms and other lip treatment products demonstrating new colors, textures, and overall compositions are regularly reformulated for new trends and new generations of users.

For the most part, prior art lip treatment products have been provided for aesthetic purposes and, additionally or alternatively, to soothe and smooth the user's lips. Functionally speaking, therefore, there has been little real separation between the many lip balms and other lip treatments taught by even the voluminous prior art. Few, if any, disclosed lip products provide or even seek to provide exceptional, perceivable sensations and feelings to the lips of the user. Indeed, it can be difficult even for experienced users to distinguish between the many lip treatments of the prior art.

Bearing the botanical name Zanthoxylum armatum, the Winged Prickly Ash is a member of the Citrus family Rutaceae. The tree is indigenous to Eastern Asia to China along the Himalayas and few other parts of the Indian subcontinent. It typically grows within the altitudinal range of 1100 to 2500 meters. It is known to extract the essential oil from the seeds of the tree through a steam distillation method. Under the prior art, Zanthoxylum oil has been used as an analgesic, antibiotic, antiseptic, stimulant. The oil has also been applied to numerous other applications, including in the attempted treatment of stress-related conditions, such as headaches, insomnia, and nervous tension, and it has been used in the treatment of circulatory issues, arthritis, inflamed joints, muscular pains, rheumatism, and sprains.

With U.S. Pat. No. 6,899,901 and U.S. Application Publication No. 2003/0215532, Nakatsu et al. discloses a sensate composition that includes a cooling sensate, a warming sensate, and a tingling sensate. Nakatsu recognizes that a tingling, numbing, or stinging sensation can be produced by, among other things, Japanese pepper extract (Zanthoxylum peperitum) having the active ingredient(s) known as Saanshool-I, Saanshool-II and Sanshoamide. Nakatsu teaches that the sensate composition could be from 0.001 to 20% by weight of an entire product composition and claims that sanshool-I, sanshool II, sanshoamide, and Spilanthol could comprise 0.001% by weight to about 10% by weight of a tingling sensate substance.

Among the many product compositions that Nakatsu briefly references are lipsticks. However, Nakatsu provides little or no disclosure of the use of Zanthoxylum peperitum as an oil. Moreover, Nakatsu has no teaching of how Zanthoxylum oil might be extracted. With its broad ranges and numerous possible combinations, Nakatsu does not teach what range of Zanthoxylum in any form might be preferred in a lip composition other than to indicate that the overall “sensate composition may be added to a product in any amount, as long as the effect of the composition is present.”

In creating a lip composition that has actual commercial viability with a desired buzzing effect while remaining enjoyable and pleasant in application and use, the present inventor has appreciated that proposing the inclusion of Zanthoxylum in a product composition, even in relation to a lip composition, is merely the beginning of the inquiry. The form of the Zanthoxylum is critical to achieving the inventor's desired buzzing effect; incorporating Zanthoxylum in its oil form is pivotal. Even more particularly, the present inventor has recognized further that the method by which the Zanthoxylum oil is extracted can prove to have significant effect on the nature and quality of the resulting oil, the buzzing effect to be achieved, and the interaction between the Zanthoxylum essential oil and soothing and moisturizing components of an overall composition.

The present inventor has thus recognized that it would be advantageous to provide a lip treatment that relies on Zanthoxylum in oil form, extracted in a manner providing preferred quality and interaction, and, potentially, at a desired level to produce a unique, buzzing sensation to the lips of the user while remaining enjoyable and pleasant such that the user realizes a phenomenal lip treatment experience.

### SUMMARY OF THE INVENTION

With a knowledge of the state of the art and the limitations and deficiencies thereof as summarized above, the present inventor set forth with the basic object of providing a lip treatment composition that presents a viable solution to the disadvantages of the prior art while providing a plurality of further improvements thereover.

A more particular object of embodiments of the invention is to provide a lip treatment of unique composition and performance that provides a perceptible tingling sensation to the mucosa membrane, such as the lips, of the user as when applied in a coating.

A more particular object of embodiments of the invention is to provide a lip treatment composition formed entirely from natural ingredients.

A further object of embodiments of the invention is to provide a lip treatment composition with one or more active ingredients that, when applied directly to the lips or other mucosa membrane, creates a buzzing sensation, potentially a perception of neurological confusion, that induces a tingling sensation across the surface of the lips.

A more particular object of embodiments of the invention is to provide a lip treatment composition that creates a buzzing sensation across the surface of the lips that may vary for different individuals but that typically begins within a short time period after application of the composition and persists for an appreciable time period thereafter.

A further object of embodiments of the invention is to provide a lip treatment composition that creates a sensation reminiscent of electrical buzzing and pulsing.

Still another object of embodiments of the invention is to provide a lip treatment composition that creates a sensation reminiscent of electrical buzzing and pulsing that intensifies in proportion to the amount of composition applied directly to the lips.

Still another object of embodiments of the invention is to provide a lip treatment composition capable of sharing and partially transferring the induced buzzing sensation to other persons by physical contact, such as kissing.

These and further objects and advantages of embodiments of the invention will become obvious not only to one who reviews the present specification but also to one who has an opportunity to make use of an embodiment of the lip treatment composition disclosed herein. It will be appreciated, however, that, although the accomplishment of each of the foregoing objects in a single embodiment of the invention may be possible and indeed preferred, not all embodiments will seek or need to accomplish each and every potential object and advantage. Nonetheless, all such embodiments should be considered within the scope of the invention.

In one embodiment, a lip treatment composition capable of producing a perceptible buzzing sensation when applied to mucosa membrane of a user comprises one or more base ingredients in combination with at least one active ingredient selected to induce a perceptible buzzing sensation when the lip treatment composition is applied to the mucosa membrane of the user. The buzzing sensation may vary in specific timing and effect for different individuals. The sensation can begin within a short time period after application of the lip treatment and can persist for an appreciable time period thereafter. In certain practices of the invention, the perceived sensation can be reminiscent of electrical buzzing and pulsing, and that sensation can intensify in proportion to the amount of composition applied. The disclosed lip treatment composition could be shared, such as by physical contact between the lips of a user of the composition and the lips of a second person.

The at least one active ingredient could comprise Zanthoxylum, potentially in the form of Zanthoxylum essential oil. Where the at least one active ingredient comprises Zanthoxylum essential oil, it could comprise between approximately 2.0000% and 4.0000% by weight of the composition. Even more particularly, Zanthoxylum essential oil could comprise approximately 3.3000% by weight of the composition. In certain practices of the invention, the at least one active ingredient could further comprise ginger essential oil and cinnamon bark oil. As where the at least one active ingredient comprises Zanthoxylum essential oil, the perceptible tingling sensation can be in the form of a perception of an electrical buzzing and pulsing sensation on mucosa membrane to which the lip treatment composition is applied.

While the lip treatment composition could comprise a lipstick, a lip gloss, or any other lip treatment, embodiments of the lip treatment composition are contemplated as comprising a lip balm. In such practices of the invention, the one or more base ingredients could include white beeswax. In any event, the lip treatment composition could further comprise at least one flavoring ingredient and, potentially, at least one coloring ingredient.

The lip treatment composition could be formed under a process comprising the steps of heating the one or more base ingredients, adding the at least one active ingredient to the one or more base ingredients after the step of heating the one or more base ingredients, and mixing the one or more base ingredients and the at least one active ingredient. The step of mixing the one or more base ingredients and the at least one active ingredient could, for instance, be performed in a rotational mixer. The method could further comprise heating the one or more base ingredients and the at least one active ingredient after the step of mixing prior to separation and packaging of the composition so formed.

One will appreciate that the foregoing discussion broadly outlines the more important features of the invention merely to enable a better understanding of the detailed description that follows and to instill a better appreciation of the inventor's contribution to the art. Before an embodiment of the invention is explained in detail, it must be made clear that the following details and descriptions of inventive concepts are mere examples of the many possible manifestations of the invention.

### DETAILED DESCRIPTION OF PREFERRED EMBODIMENTS

The lip treatment composition disclosed herein is subject to varied embodiments, each within the scope of the invention. However, to ensure that one skilled in the art will be able to understand and, in appropriate cases, practice the present invention, certain preferred embodiments of the broader invention revealed herein are described below.

With the foregoing in mind, manifestations of the lip treatment composition pursuant to the present invention could, by way of example and not limitation, be founded on base ingredients, including flavors. The base ingredients could be standard and commonly used for the type of lip treatment composition at hand. It will again be understood that the lip treatment composition could comprise substantially any type of lip treatment composition, including but not limited to lip balm, a lipstick, lip gloss, or some other lip coating.

One presently contemplated composition embodying the invention takes the form of a lip balm. There, the lip treatment composition could be formed with base ingredients, such as white beeswax and sweet almond oil. For example, the white beeswax could be provided at approximately 20.0000% by weight, and the sweet almond oil could be provided at approximately 66.5000% by weight.

The present inventor has devised of an active ingredient combination that is particularly effective in producing the desired buzzing sensation while remaining pleasant and enjoyable to the user. As taught herein, the active ingredients can be considered to be founded on Zanthoxylum, provided in essential oil form, for producing a desired physical sensation reminiscent of electrical buzzing. In the composition disclosed herein, the Zanthoxylum is provided in essential oil form. Moreover, the Zanthoxylum oil is extracted using carbon dioxide in liquid form as the solvent, which has been found by the inventor to produce Zanthoxylum oil in a high quality form in relation to the present invention's desired buzzing effect while combining well in practice and resulting effect with other active and inactive ingredients. When carbon dioxide is converted to liquid by using low temperatures and high pressure, it is operative as a safe and effective solvent that allows all the desirable active constituents of the botanical to be collected without the risk of heat degradation. Once the extraction is complete, the carbon dioxide is allowed to return to its natural gaseous state, leaving behind only the extracted essence of the plant. The carbon dioxide extraction process and the extracted Zanthoxylum oil component used herein provides a qualitatively different effect and capacity for integration with other active and inactive ingredients than that of the known prior art. The form of Zanthoxylum oil so extracted thus yields uniform and replicable potency at the percentage ranges disclosed herein distinctive from the naturally-occurring source from which it is extracted.

The Zanthoxylum essential oil could, in preferred embodiments, be included at between approximately 2.0000% and 4.0000% by weight. For the desired buzzing effect of the present invention, the Zanthoxylum essential oil may most preferably be provided at approximately 3.3000% by weight. The active ingredients could further include one or more of ginger essential oil, cinnamon bark oil, sweet orange extract, vitamin E, and rosemary oil extract. For instance, additional active ingredients could be 0.5000% ginger essential oil, 0.4000% cinnamon bark oil 18J, 0.5000% sweet orange extract, 0.2000% T50 natural vitamin E, and 0.1000% rosemary oil extract, all by weight.

The foregoing combination of the engineered Zanthoxylum essential oil at an unexpectedly effective non-naturally occurring percentage by weight with the disclosed active and inactive ingredients in specified ranges does not exist in nature. Moreover, providing such an engineered Zanthoxylum essential oil at a specified percentage by weight and combined with an acceptable carrier base ingredient further departs from what could be considered to be occurring naturally.

The specific range of Zanthoxylum essential oil extracted by a liquid carbon dioxide extraction process in the composition of between approximately 2.0000% and 4.0000% by weight produces unexpected, advantageous results. Moreover, the precise percentage of Zanthoxylum essential oil of 3.3000% by weight of the composition is particularly advantageous and produces unexpected results at a percentage outside of the ranges of the known prior art. The specified range and percentage of Zanthoxylum essential oil is found to produce the ideal, strong buzzing effect without an undesirable burning effect. Conversely, the simple inclusion of Zanthoxylum in some other form and not extracted in a liquid carbon dioxide process diminishes the desired effect, such as by ineffective buzzing, undesirable burning, or ineffective combination with other components. The disclosed range and percentage by weight of Zanthoxylum essential oil has been found to produce a lip treatment composition with uniform and replicable potency.

Providing the Zanthoxylum essential oil within the desired percentage range permits the additional inclusion of percentages of moisturizing oils that are desired in a quality lip formula. The combination of the buzzing oil along with soothing and moisturizing oils makes the disclosed formulation effective for its intended purpose while being enjoyable and pleasant in application and usage.

The specific range of Zanthoxylum essential oil in the composition of between approximately 2.0000% and 4.0000% by weight produces unexpected, advantageous results. The precise percentage of Zanthoxylum essential oil of 3.3000% by weight of the composition is particularly advantageous and produces unexpected results at a percentage outside of the ranges of the prior art. The specified range and percentage of Zanthoxylum essential oil is found to produce the ideal, strong buzzing effect without an undesirable burning effect. Zanthoxylum essential oil produced through liquid carbon dioxide extraction in combination with the present composition's remaining active and inactive ingredients are different in identity compared with what exists in nature. The Zanthoxylum essential oil so extracted has a distinctive identity in comparison to the naturally-occurring tree from which it is extracted.

The composition could further include flavoring and coloring ingredients. By way of example and not limitation, a lip treatment composition as taught herein could be a strawberry composition. To achieve a composition that might be characterized as a strawberry composition, such an embodiment could further include approximately strawberry flavoring and red coloring. For example, the flavoring ingredient could be 8.000% by weight strawberry 416041SU R517913 (natural), and the coloring could be approximately 0.5000% by weight red oil-based candy color. Of course, strawberry flavoring and corresponding coloring are mere examples. Other flavors and colorings will be readily possible using the foregoing teachings and should be considered as part of this disclosure.

So disclosed, the lip treatment composition according to the invention can be functional for its intended purpose of producing an effective yet enjoyable buzzing effect without a need for menthol or mint. It will be understood, though, that one or more such ingredients could be included within the scope of the invention and should not be considered as excluded except as might be expressly required by the claims.

It will be understood that the lip treatment composition could include additional active or inactive ingredients. Accordingly, although the foregoing components and proportions of components may indeed be preferable and advantageous in achieving one or more objects of the invention, the lip treatment composition shall not be interpreted to require all of the foregoing components, to be limited to the specified components, or to be limited even to the approximate proportions of components except as the claims might expressly specify. By way of example, fewer, additional, or different ingredients or ingredients in different proportions could be provided to create different flavors or effects or otherwise within the scope of the invention.

Finished lip treatment compositions could be created according to a particularized process. The process could begin with heating the base ingredient(s), such as the white beeswax, which again could comprise roughly 20.0000% by volume of the overall composition, to melting. The ingredients of the lip treatment composition can then be combined, potentially while continuously or periodically mixing the components. For instance, the components could be mixed in a rotational mixer. The mixer could, for example, spin at a rate of approximately 300 revolutions per minute (RPM). With the components rotating at a given rotational rate, such as approximately 300 RPM, the composition can be heated to an elevated temperature, such as approximately 60° C. (140° F.). So combined and heated, the composition can be dispensed to suitable containers for distribution and use or, potentially, for further separating and packaging.

The lip treatment composition so disclosed can be applied by a user to the mucosa membrane, such as the lips, of a human. The lip treatment composition seeks to provide unique performance with a perceptible tingling sensation to the lips of the user, potentially while being formed entirely from natural ingredients. The lip treatment composition has one or more active ingredients that, when applied directly to the lips or other mucosa membrane, seeks to create a sensation reminiscent of electrical buzzing across the surface of the lips.

Although the buzzing sensation may vary in specific timing and effect for different individuals, the sensation typically begins within a short time period after application of the lip treatment and persists for an appreciable time period thereafter. By way of example and not limitation, the sensation could begin within approximately thirty to ninety seconds of application and can last for approximately ten to fifteen minutes. The perceived sensation can be characterized as being reminiscent of electrical buzzing and pulsing, and that sensation can intensify in proportion to the amount of composition applied directly to the lips. Moreover, the lip treatment composition disclosed herein is capable of permitting sharing and partially transferring the induced sensation to other persons by physical contact between the lips or another body surface of a first person to which the composition has been applied and the lips or another body surface of a second person. For example, the composition and its effect could be transferred by kissing.

With certain details and embodiments of the present invention for a lip treatment composition disclosed, it will be appreciated by one skilled in the art that numerous changes and additions could be made thereto without deviating from the spirit or scope of the invention. This is particularly true when one bears in mind that the presently preferred embodiments merely exemplify the broader invention revealed herein. Accordingly, it will be clear that those with major features of the invention in mind could craft embodiments that incorporate those major features while not incorporating all of the features included in the preferred embodiments.

Therefore, the following claims shall define the scope of protection to be afforded to the patent holder. Those claims shall be deemed to include equivalent constructions insofar as they do not depart from the spirit and scope of the invention. It must be further noted that a plurality of the following claims may express certain elements as means for performing a specific function, at times without the recital of structure or material. As the law demands, any such claims shall be construed to cover not only the corresponding structure and material expressly described in this specification but also all equivalents thereof.

I claim as deserving the protection of Letters Patent:

1. A lip treatment composition for creating a perceptible buzzing sensation when applied to mucosa membrane of a user, the lip treatment composition comprising:
one or more base ingredients; and
at least one active ingredient comprising Zanthoxylum essential oil extracted in a liquid carbon dioxide process to induce a perceptible buzzing sensation when the lip treatment composition is applied to the mucosa membrane of the user.

2. The lip treatment composition of claim 1 wherein the at least one active ingredient further comprises ginger essential oil and cinnamon bark oil.

3. The lip treatment composition of claim 1 wherein the Zanthoxylum essential oil comprises between approximately 2.0000% and 4.0000% by weight of the composition.

4. The lip treatment composition of claim 3 wherein the Zanthoxylum essential oil comprises approximately 3.3000% by weight of the composition.

5. The lip treatment composition of claim 1 wherein the lip treatment composition comprises a lip balm and wherein the one or more base ingredients include white beeswax.

6. The lip treatment composition of claim 1 further comprising at least one flavoring ingredient and at least one coloring ingredient.

7. The lip treatment composition of claim 1 wherein the lip treatment composition is formed under a process comprising the following steps:
providing one or more base ingredients;
providing at least one active ingredient comprising Zanthoxylum essential oil extracted in a liquid carbon dioxide process to induce a perceptible buzzing sensation when the lip treatment composition is applied to the mucosa membrane of the user;
heating the one or more base ingredients;
adding the Zanthoxylum essential oil to the one or more base ingredients after the step of heating the one or more base ingredients; and
mixing the one or more base ingredients and the at least one active ingredient.

8. The lip treatment composition of claim 7 wherein the step of mixing the one or more base ingredients and the at least one active ingredient is performed in a rotational mixer.

9. The lip treatment composition of claim 8 further comprising the step of heating the one or more base ingredients and the at least one active ingredient after the step of mixing.
